(19) United States
(12) Patent Application Publication
(10) Pub. No.: US 2004/0038910 A1
(43) Pub. Date: Feb. 26, 2004

(54) Remedy for diabetes mellitus

(76) Inventors: Makoto Fujii, Kagoshima (JP); Susumu Hizukuri, Kagoshima (JP)

Correspondence Address: KOLISCH HARTWELL, P.C., 520 S.W. YAMHILL STREET, SUITE 200, PORTLAND, OR, 97204, US

(21) Appl. No.: 10/416,748

(22) PCT Filed: Oct. 31, 2001
(86) PCT No.: PCT/JP01/09542

(30) Foreign Application Priority Data
Nov. 15, 2000 (JP) 2000-347372

(51) Int. Cl. A61K 31/70; A61K 31/7012

(52) U.S. Cl. 514/23; 514/53

(57) ABSTRACT

A remedy for diabetes mellitus characterized in that it contains L-arabinose and sucrose (sugar or a sugar-containing food or beverage) as effective ingredients. A food or beverage for improvement of diabetes mellitus characterized in that it contains L-arabinose and sucrose (sugar or a sugar-containing food or beverage) as effective ingredients. A method for using L-arabinose characterized in that, for the treatment of diabetes mellitus, L-arabinose is taken together with or prior to ingestion of sucrose (sugar or sugar-containing food or beverage).

### TECHNICAL FIELD

The present invention relates to the use of L-arabinose for the treatment of diabetes mellitus. The invention further relates to remedies for diabetes mellitus in which the interaction of L-arabinose with sucrose is utilized, with food or beverages, for improvement of diabetes mellitus.

### BACKGROUND

Although the majority of saccharides in food are starch, a lot of sucrose is consumed as the most preferred sweetener throughout the world. In 1996 for example, 87 g and 185 g of sucrose/person/day were consumed in Japan and in the United States, respectively. Ingestion of large amounts of sucrose tends to induce hyperglycemia and obesity.

Obesity is a well-known danger factor for non-insulin-dependent diabetes mellitus (NIDDM). Many diabetics are persons suffering from NIDDM. In addition to the administration of insulin or administration of an SU agent which accelerates the secretion of insulin, administration of an inhibitor of α-glucosidase, etc. is available for the therapy of NIDDM. The former two agents promote the ingestion of saccharide from blood into cells whereby blood-sugar level is lowered while, in the latter, action of an enzyme (glucosidase) which digests and decomposes the saccharide is suppressed and digestion and absorption of the saccharide in the small intestine is delayed and/or suppressed to inhibit the ingestion of the saccharide from the intestine into the blood, whereupon a rise in blood-sugar level after meals is suppressed.

On the other hand, L-arabinose has been known to have an action of inhibition of sucrase, which is a sucrose-decomposing enzyme in the small intestine, and it has been recognized that such an action of L-arabinose can be utilized to partially inhibit the decomposition of sucrose and to reduce the energy of sucrose. For example, in the Japanese Patent Laid-Open No. Hei-6/65080, there is disclosed that L-arabinose has an action of suppressing the rise of blood-sugar level in mice during 30 to 120 minutes after loading with sucrose. However, the above application disclosed only the above-mentioned action of sucrase inhibition of L-arabinose, with its action of suppressing increases in blood-sugar level after meals, and a suppression of body weight increase caused thereby. The efficacy of improvement and treatment of diabetes mellitus by lowering the blood-sugar level using L-arabinose is not made clear.

The present invention has been invented in view of the current state of the prior art as such and its object is to provide a remedy for diabetes mellitus effective to diabetic patients utilizing L-arabinose, which results in hypoglycemic action, and food or beverages for improvement of diabetes mellitus and a method for using L-arabinose for the therapy of diabetes mellitus.

In order to achieve such an object, the present inventors have carried out various studies and have unexpectedly found that, although the use of L-arabinose alone results in only a transient suppressive action on hyperglycemia due to its sucrase-inhibiting action, blood-sugar levels of KK-A^{Y} mice (used to model non-insulin-dependent diabetes mellitus) are drastically lowered and diabetes mellitus is improved and cured when the mice are continuously administered feed in which L-arabinose is mixed in a specific ratio with sucrose, which is otherwise a causative agent for diabetes mellitus.

### SUMMARY

Thus, the present invention relates to a remedy for diabetes mellitus which is characterized by containing L-arabinose and sucrose (for example, sugar or sugar-containing food or beverages are used as a source for sucrose) as effective ingredients.

The present invention further relates to food or beverages for improvement of diabetes mellitus, which are characterized by containing L-arabinose and sucrose (for example, sugar or sugar-containing food or beverages are used as a source for sucrose) as effective ingredients.

The present invention still further relates to a method for using L-arabinose, which is characterized in that, for the treatment of diabetes mellitus, L-arabinose is taken together with or prior to ingestion of sucrose (for example, sugar or sugar-containing food or beverages are used as a source for sucrose).

### BRIEF DESCRIPTION OF THE DRAWINGS

FIG. 1 shows the changes in blood-sugar level (mg/dl) during the study period of group A and group B for 30 days, as described in Example 1.

FIG. 2 shows the changes in blood-sugar level (mg/dl) during the study period of group C and group D for 30 days, as described in Reference Example 1.

### DETAILED DESCRIPTION OF THE PREFERRED EMBODIMENTS

A remedy for diabetes mellitus, and food or beverages for the improvement of diabetes mellitus, according to the present invention will be specifically illustrated hereinafter, although the present invention is not limited thereto.

With regard to L-arabinose used in the present invention, that which is manufactured by various known methods can be used. For example, there may be used that which is manufactured in high purity and in high efficiency where plant fiber containing L-arabinose as a part of the constituting saccharides is subjected to acid hydrolysis using a low concentration of acid, whereupon L-arabinose is selectively produced as disclosed, for example, in Japanese Patent Laid-Open No. Hei-11/313700.

With regard to sucrose used in the present invention, there may be used chemically purified sucrose as well as a sucrose-containing substance as a source for sucrose such as sugar or sugar-containing food or beverage. There are various sugar products depending upon material, manufacturing method, degree of purification, color, processed form, etc. thereof, and any of them may be used in the present invention so far as it contains sucrose.

With regard to the remedy for diabetes mellitus and the food or beverages for improvement of diabetes mellitus according to the present invention, L-arabinose and sucrose may be ingested separately, with a time interval in between, provided that the ingestion results in a state wherein L-arabinose and sucrose are simultaneously present in the intestine. However, since L-arabinose is slowly absorbed from the intestinal tract and therefore remains for a long time in the intestine, while sucrose shows quick digestion and absorption, it is effective for the therapy or the improvement of diabetes mellitus that L-arabinose is ingested together with, or prior to, ingestion of sucrose (sugar or sugar-containing food or beverages). In the remedy for diabetes mellitus of the present invention, or with respect to the food or beverages for improvement of diabetes mellitus according to the present invention, it is preferred that the L-arabinose be present in a ratio of 0.5˜90% by weight or, preferably, 1˜50% by weight, to sucrose. When the amount of L-arabinose used is less than the lower limit given above, it is not preferred because the effect of the joint use of L-arabinose with sucrose is hardly expressed while, when the amount is greater than the above upper limit, it is not preferred in view of economy, since the greater amount cannot be expected to produce an additional effect.

In addition to the use of L-arabinose and sucrose only, the remedy for diabetes mellitus according to the present invention may also be easily manufactured by combining L-arabinose and sucrose with known pharmaceutical carriers to create pharmaceutical preparations. Manufacture of such pharmaceutical preparations is usually conducted in such a manner that L-arabinose and sucrose are compounded with pharmaceutically acceptable liquid or solid carriers and, if necessary, solvent, dispersing agent, emulsifier, buffer, stabilizer, filler, binder, disintegrating agent, lubricant, etc. are added thereto in order to give solid preparations such as tablets, granules, diluted powders, powders, and capsules, or liquid preparations such as normal liquids, suspensions, and emulsions. It is also possible to prepare a dried preparation which can be made into liquid by addition of an appropriate carrier thereto before use.

It is preferred that the remedy for diabetes mellitus according to the present invention be an oral preparation so as to effectively express the effect by the joint use of L-arabinose and sucrose. With regard to a pharmaceutical carrier for the oral preparation, there may be used, for example, starch, lactose, white sugar, mannitol, carboxymethyl cellulose, corn starch, and/or inorganic salts. For the manufacture of the oral preparation, it is also possible to further compound one or more binders, disintegrating agents, surface-active agents, lubricants, fluidity promoters, flavor corrigents, coloring agents, etc. therewith.

Dosage of the remedy for diabetes mellitus according to the present invention may be appropriately determined depending upon dosage form, administering method, object of use and age, body weight, and symptoms of the patient applied therewith and, although it is not definite, the amount of L-arabinose contained in the preparation is usually from 1 mg to 10 g/kg per day for adults. It goes without saying that the dose varies depending upon various conditions and, in some cases, a smaller dose than given in the above range may be sufficient while, in some other cases, a greater dose than that given in the above range may be necessary. The remedy for diabetes mellitus according to the present invention may be ingested daily by adding to any food or beverage, as well as by oral administration.

Although there is no particular limitation for the manufacturing method of the food or beverage for improvement of diabetes mellitus according to the present invention, there may be exemplified the manufacture by means of cooking, processing, and commonly used manufacturing methods for food or beverages, provided that the manufactured food or beverage contains L-arabinose and sucrose having an improving action for diabetes mellitus as an effective ingredient. With regard to the food or beverage for improvement of diabetes mellitus according to the present invention, there is no particular limitation for its form as long as sucrose and L-arabinose having an improving action for diabetes mellitus are contained therein, added thereto, and/or diluted therein, and such food or beverage includes orally applicable forms such as tablets, granules, capsules, gels, and sols.

The reason why the joint use of L-arabinose and sucrose in the remedy for diabetes mellitus and the food or beverage for improving diabetes mellitus according to the present invention is effective for treatment and improvement of diabetes mellitus has not been clearly elucidated. Without wishing to be bound by theory, it is judged that numbers of specific enterobacteria are increased or activated due to the presence of L-arabinose and sucrose at the same time in the intestine and that those bacteria produce a substance which is effective for reducing sugar-blood level by promoting the incorporation of saccharide from blood into cells.

### EXAMPLES

The effect of the joint use of L-arabinose and sucrose used in the present invention for the therapy or improvement of diabetes mellitus will be illustrated as hereunder.

### Example 1

Twenty model mice for non-insulin-depending diabetes mellitus showing hyperglycemia (260˜270 mg/liter) (KK-A^{y}, male, 8 weeks age, Nippon Clair) were preliminarily raised for one week, divided into two groups (group A and group B) and used for the test. Each of the mice was placed in a plastic cage separately and raised by keeping at 23±1° C. under a 12-hour bright-and-dark cycle (bright environment from 7 a.m. to 7 p.m. and dark environment from 7 p.m. to 7 a.m. of the next day) where feed and water were freely taken by the mice. Blood-sugar level (concentration of glucose in blood) was measured by a glucose oxidase method after collecting the blood from the venous plexus of the eyeground at 9 a.m.

The feed A mentioned in Table 1 (a feed containing α-corn starch, sucrose, and cellulose as carbohydrate sources but containing no L-arabinose) was given to the mice of group A while the feed B mentioned in Table 1 (a feed containing α-com starch, sucrose, and cellulose as carbohydrate sources and also containing L-arabinose) was given to the mice of group B and the study was carried out for 30 days.

TABLE 1
Composition of the Feed

| | |
|---|---|
| Composition | Feed |

| (% by weight) | A | B | C | D |
|---|---|---|---|---|
| α-Corn starch | 35.85 | 35.85 | 55.85 | 55.85 |
| Sucrose | 20 | 20 | 0 | 0 |
| L-arabinose^{*1} | 0 | 2.5 | 0 | 2.5 |
| Cellulose | 5.0 | 2.5 | 5.0 | 2.5 |
| Corn oil | 6 | 6 | 6 | 6 |
| Mixture of minerals^{*2} | 6 | 6 | 6 | 6 |
| Mixture of vitamins^{*2} | 2 | 2 | 2 | 2 |
| Choline chloride | 0.15 | 0.15 | 0.15 | 0.15 |
| Casein | 25 | 25 | 25 | 25 |

^{*1}L-arabinose (purity: 98%) manufactured by Sanwa Dempun Kogyo K. K.
^{*2}manufactured by Oriental Yeast K. K.

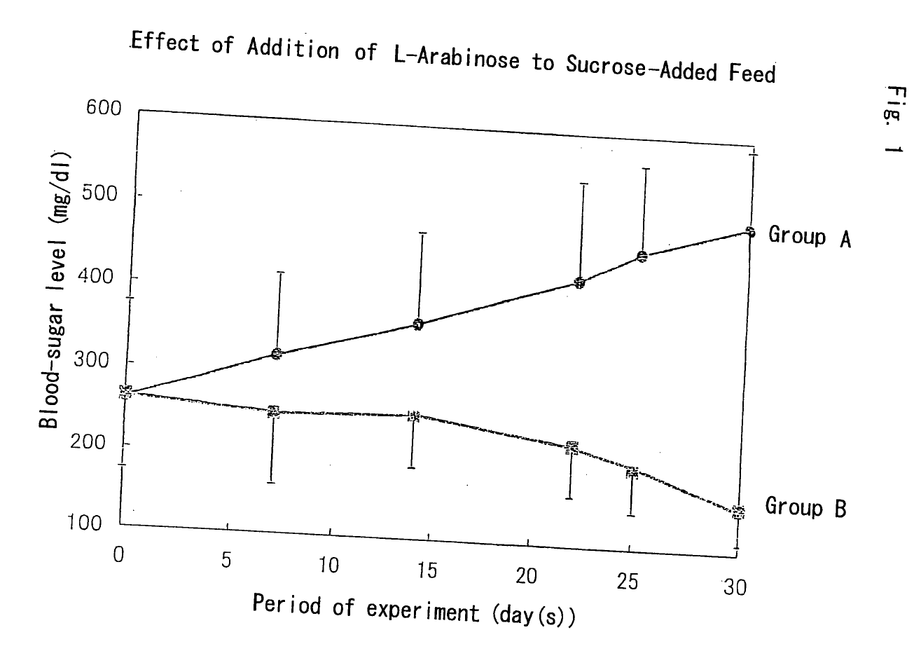

Changes in blood-sugar level (mg/dl) of the groups A and B raised for the study period of 30 days are shown in FIG. 1. As will be apparent from FIG. 1, in the group A where the mice were given feed A containing sucrose but containing no L-arabinose, blood-sugar level continued to increase from the 7th day while, in the group B where the mice were given feed B containing sucrose and also containing L-arabinose, lower blood-sugar levels than that determined for group A were continuously noted from the 7th day, lower blood-sugar levels than that at the start of the experiment were noted after the 21st day and, from the 25th day until 30th day, a continuous decrease in the blood-sugar levels was apparently noted, whereupon an effect of improving and treating diabetes mellitus was expressed.

The result of Example 1 shows an effect of improving and treating the blood-sugar level is expressed as the joint action by simultaneous administration of sucrose and L-arabinose but, only by way of Example 1, there is still a possibility that addition of L-arabinose only may have the same effect. In order to make the possibility clear, an experiment was carried out in the following Reference Example 1 where no sucrose was added to the feed, alternatively, α-corn starch was added instead of sucrose.

### Reference Example 1

As in Example 1, a feed C mentioned in Table 1 (containing α-corn starch and cellulose as carbohydrate sources but containing neither sucrose nor L-arabinose) was given to the mice of group C, while a feed D mentioned in Table 1 (containing α-corn starch and cellulose as carbohydrate sources and also containing L-arabinose but containing no sucrose) was given to the mice of group D, and the study was carried out for 30 days.

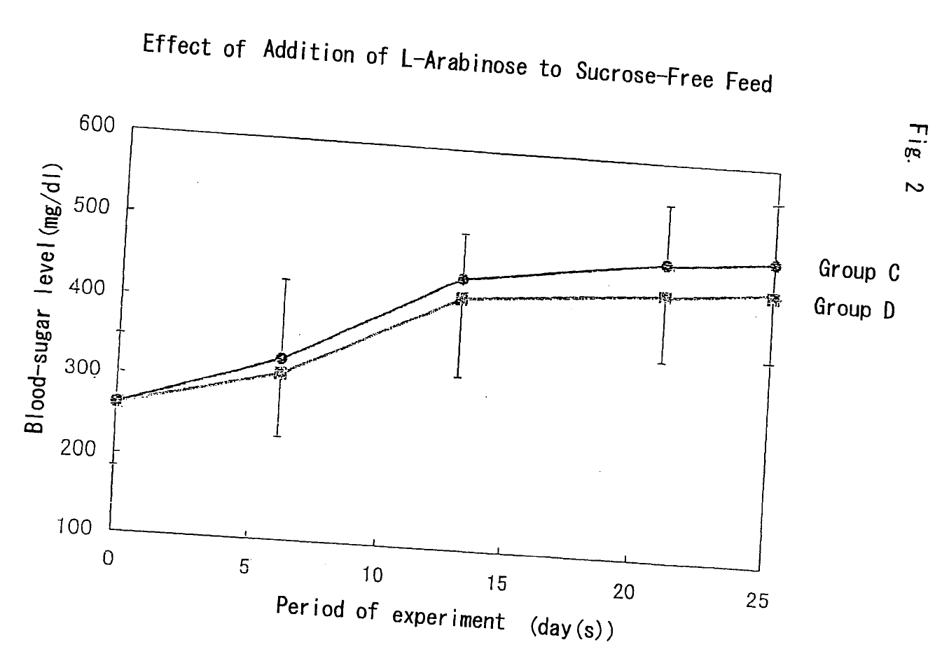

Changes in blood-sugar level (mg/dl) of the groups C and D were studied for 30 days as shown in FIG. 2. As will be apparent from FIG. 2, mice of both groups C and D continued to exhibit increasing blood-sugar levels, as in the case of the group A of Example 1 during a period of the experiment whereby no improving effect in blood-sugar levels was noted. That shows that no decrease in blood-sugar level takes place when only L-arabinose is given without addition of sucrose. Now, results of Example 1 and Reference Example 1 show that high blood-sugar level in model mice for non-insulin-independent diabetes mellitus can be continuously lowered and improved by a simultaneous ingestion of a mixture of sucrose and L-arabinose, showing that such mixture is an effective remedy for diabetes mellitus.

Incidentally, it is not always necessary that sucrose and L-arabinose are administered as a mixture but, for example, with regard to sucrose, food or beverages containing sucrose may be ingested separately. In that case, L-arabinose may be ingested either before or after sucrose, although the early ingestion is effective.

### Advantage of the Invention

In accordance with the present invention, a continuous decrease in blood-sugar level can be achieved by a joint use of sucrose and L-arabinose and that is very useful as a remedy for diabetes mellitus and as a food or beverage for improvement of diabetes mellitus.

What is claimed is:

1. A remedy for diabetes mellitus characterized in that it contains L-arabinose and sucrose as effective ingredients.

2. The remedy for diabetes mellitus according to claim 1, characterized in that it is an oral preparation.

3. The remedy for diabetes mellitus according to claim 1, characterized in that sugar or a sugar-containing food or beverage is used as a source of sucrose.

4. The remedy for diabetes mellitus according to claims 1, characterized in that L-arabinose is present in a ratio of about 0.5% to about 90% by weight to sucrose.

5. A food or beverage for improvement of diabetes mellitus characterized in that it contains L-arabinose and sucrose.

6. The food or beverage for improvement of diabetes mellitus according to claim 5, characterized in that the L-arabinose is present in a ratio of about 0.5% to about 90% by weight to sucrose.

7. A method for using L-arabinose characterized in that, for the therapy of diabetes mellitus, L-arabinose is taken together with sucrose, or prior to ingestion of sucrose.

8. The method for using L-arabinose according to claim 7 characterized in that sugar or a sugar-containing food or beverage is used as a source for the sucrose.
